(19) United States
(12) Patent Application Publication
(10) Pub. No.: US 2008/0091084 A1
(43) Pub. Date: Apr. 17, 2008

(54) USER INTERFACE FOR A PK/PD ANESTHETIC DRUG MODEL DISPLAY

(76) Inventors: Laurence M. Yudkovitch, Milwaukee, WI (US); Ronald P. Makin, Fitchburg, WI (US); Rene Coffeng, Helinski (FI); Toni Taiminen, Vantaa (FI); Tom J. Haggblom, Vantaa (FI); Jussi Heikkola, Espoo (FI)

Correspondence Address: ANDRUS, SCEALES, STARKE & SAWALL, LLP, 100 EAST WISCONSIN AVENUE, SUITE 1100, MILWAUKEE, WI, 53202, US

(21) Appl. No.: 11/564,005

(22) Filed: Nov. 28, 2006

Related U.S. Application Data
(60) Provisional application No. 60/851,109, filed on Oct. 12, 2006.

(51) Int. Cl. G06F 19/00; A61B 5/00

(52) U.S. Cl. 600/300; 702/19

(57) ABSTRACT

A graphical user interface for use by a clinician in the monitoring of the depth of the anesthesia of a patient. The graphical user interface comprising a drug library with data related to a plurality of anesthetic drugs, a drug administration window for displaying drug administration data, at least one window disposed for the display of a pharmacokinetic graph and a pharmacodynamic graph, and a scale selector comprising a first condition wherein the pharmacokinetic model and the pharmacodynamic models are displayed and a second condition where only the pharmacokinetic model is displayed.

### CROSS REFERENCE TO RELATED APPLICATION

This application claims priority under 35 USC §119(e) of the co-pending U.S. Provisional Application 60/851,109, filed on Oct. 12, 2006 and entitled “USER INTERFACE FOR A PK/PD ANESTHETIC DRUG MODEL DISPLAY.”

### FIELD OF THE INVENTION

The present invention relates to a user interface for a life support system, more specifically, the present invention relates to a user interface for an anesthetic drug model display.

### BACKGROUND OF THE INVENTION

In the operating room, the anesthesiologist needs to assess the patient's condition and adjust the therapy using a wide variety of distinct medical devices. These devices often have limited communication abilities between each other, resulting in an incomplete depiction of the patient's condition to the anesthesiologist. A clinical must therefore mentally keep track of the patient's level of sedation, analgesia, and relaxation, the three physiological components of anesthesia, based on the clinician's recall of the drugs that have been administered, and the clinician's own familiarity with each drug's pharmacokinetic (PK) and pharmacodynamic (PD) models.

The practice of intra-operative anesthesia typically involves administering sedative, analgesic, and neuromuscular relaxant drugs or agents to a patient. These drugs manage the patient's level of consciousness, pain management, and neuromuscular blockade. Typically, each drug has a pharmacokinetic model that specifies what the body does to the drug and a pharmacodynamic model that specifies how the drug interacts with the body. More specifically, the pharmacokinetic model represents how the drug is absorbed, distributed, and broken down by the patient's body. The pharmacodynamic model approximates the effect that the patient feels from the administration of the drug. These models are usually derived in isolation from one another and are based upon standard demographic information of the patient such as sex, age, height, and/or weight. However, in a clinical setting, multiple drugs and/or agents are typically used together. The interactions between these drugs may be additive, and produce no additional effects, may be synergistic, and produce a greater total effect than the sum of the individual drug effects, or may be antagonistic, and produce less total effect than the sum of the individual effects.

The interaction between two anesthetic drugs has been represented by three-dimensional response surfaces. These surfaces represent the probability of a non-response to a specific effect at different concentrations of the two drugs. However, these three-dimensional graphs are complex and difficult to display on a display that is typically available for the display of a graph based on an anesthetic drug model. Therefore, the challenge is to display these varying probabilities of drug interactions on a two-dimensional graph that can easily be interpreted by a clinician during anesthesia.

The display of drug pharmacokinetics and the resulting pharmacodynamics becomes still more complex when more than one pharmacodynamic effect must be displayed on the same graph. For example, when considering analgesia, one can consider varying levels of pain such as high pain (intubation) and low pain (post-op). The challenge is to display these related, but distinct effects on the same two-dimensional graph. Therefore, it is desirable that the display be able to display the pharmacokinetic information of the effect site concentration of the administered drugs, at least one pharmacodynamic effect, the probability of each displayed effect, and reference points to the probability range of those effects.

Displays have been developed in the prior art that show both the PK and PD models to the clinician in a real time display. This display, however, is limited in its ability as a clinical user interface. The prior art display only supports the generic name of drugs, which makes it difficult for clinicians who aren't always familiar with generic names, or often relying upon the common names of the drugs. Furthermore, the prior art display required the user to manually enter the concentration of the drug administered each time. This makes the system more difficult to use by clinicians who administer different concentrations of drugs throughout the same case. Further, the prior art display required that drug administration data be input into the display in real time. This often resulted in the need for an extra clinician to be present in the operating room to manually document and enter the data into the display in real time.

Additional limitations of the prior art display include user interface elements that impede a clinician's interpretation of the displayed PK and PD models. Namely, there was no indication that the pharmacodynamic effectiveness range is a non-linear scale. The lack of such an indication can potentially confuse a clinician in interpreting the PD model. Furthermore, when displaying a combinational graph comprising PK and PD models, the scaling of the graph may be required to be large due to the PD model, while the drug effect site concentration displayed in the PK model is relatively small, thus the clinician's ability to interpret the PK model is limited by the scaling needed for the display of the PD model.

Therefore, it is desirable in the present field of anesthetic drug model displays to provide a user interface that improves the ease with which the clinician can enter drug administration data, allows for the editing of the drug administration data retroactively, displays both the PK and PD models on the same graph, and addresses the scaling problems experienced in the prior art for the drug effectiveness ranges.

### SUMMARY OF THE INVENTION

A user interface for a pharmacokinetic and pharmacodynamic anesthetic drug model display is herein disclosed. In an embodiment, the user interface displays a time based display with indications of drug and fluid administration and graphs depicting pharmacokinetic models and pharmacodynamic models of the drugs administered. An embodiment the user interface further comprises a drug library that comprises frequently used drugs and standard concentrations of the drugs that are used in a medical facility. The drug library may comprise both a drug's common and generic name and upon selection of a drug and drug concentration from the drug library the drug name and the concentration may be displayed on the drug administration display.

In a further embodiment the user interface allows the editing of the drug name, drug concentration, the infusion rate or bolus amount, and administration time, or the deleting of an erroneous entry.

In a still further embodiment the user interface comprises a therapy window to aid in the interpretation of a pharmacodynamic effect model for the patient. More specifically, an embodiment of the user interface comprises a therapy window divided into equal-probability ranges, wherein the size of the range within the therapy window provides a clinician with information about the non-linearity of the pharmacodynamic effect. In an alternative embodiment, the therapy window may use a non-linear shaded gradient to depict the probability ranges of the pharmacodynamic effect.

In a further embodiment of the user interface, the scale of the graph depicting the pharmacokinetic and pharmacodynamic models may be modified between a normalized scale representing both pharmacodynamic effect and a pharmacokinetic effect site concentration.

### BRIEF DESCRIPTION OF THE DRAWINGS

FIG. 1 depicts a screen shot of an embodiment of the user interface;

FIG. 2 depicts a screen shot of an embodiment of the user interface depicting the PK only analgesia scale; and

FIG. 3 is a schematic diagram of an embodiment of a system comprising the user interface.

### DETAILED DESCRIPTION OF THE INVENTION

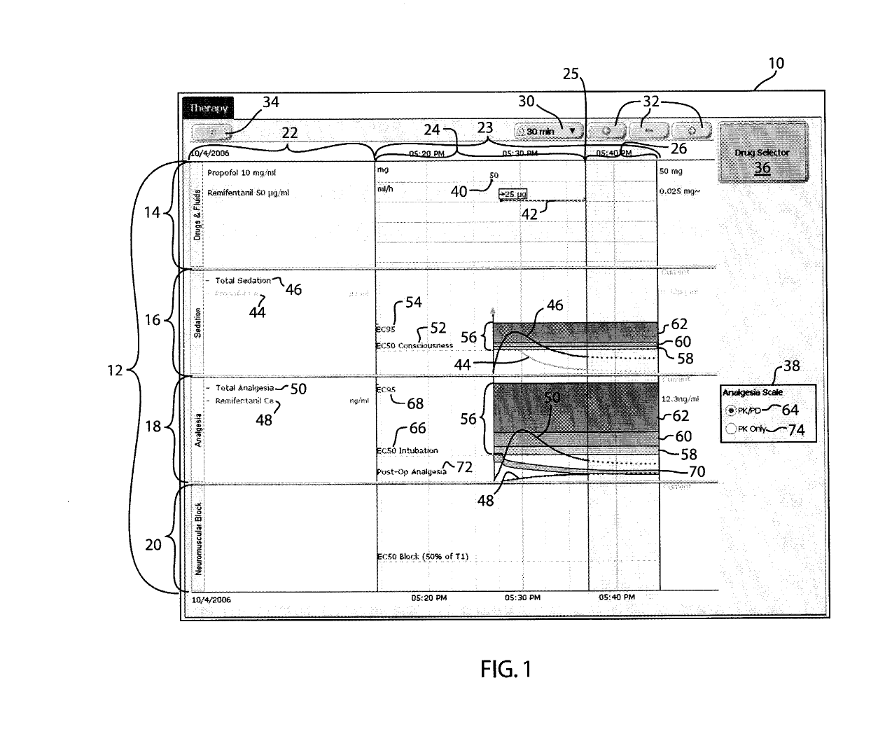

FIG. 1 depicts a screen shot of a user interface 10. The user interface 10 may be displayed on a computer display terminal (not depicted) of a clinician workstation or on any other suitable display for displaying an anesthetic drug model present in an operating room. Such a display may comprise an LCD or other such flat-screen technology. Furthermore, the workstation or display comprising the user interface 10 may be such that the clinician interacts with the user interface 10 using touch-screen technology that is activated by the clinician's finger or a stylus. Alternatively the clinician may control a cursor on the user interface 10 with an input means such as a mouse, data knob, directional pad, or keyboard; however, it is understood that any type of input means suitable for entering data into a computer workstation user interface is contemplated within the scope of the invention.

The user interface 10 may be a graphical user interface (GUI) that may comprise the entire area of the display, or may be a portion of the area of the display. The user interface 10 may further comprise a plurality of horizontal display windows 12. In an embodiment, the display windows 12 may include a drug administration window 14, a sedation window 16, a analgesia window 18, and a neuromuscular block window 20. In an embodiment of the user interface 10, each of the display windows displays the pharmacokinetic (PK) and pharmacodynamic (PD) information pertaining to a specific type of anesthetic effect.

The user interface 10 may divide the horizontal windows 12 to each comprise a vertical a drug listing region 22. The drug listing region 22 displays the name of any drug for which that window 12 is displaying data. The user interface 10 may further divide the horizontal windows 12 to each comprise a vertical graph region 23. The graph region 23 displays graphical and/or numeric data pertaining to the administered drugs. The graph region may be a time-based display. The time-based display may include a historical data region 24 and a future region 26. The historical data region 24 may be separated from the future region 26 by a present line 25 that indicates the current time in the display. The historical data region 24, depending on the window 12, may display data regarding the drugs that have been administered, or may display the PK and PD graphs for the drugs that have been administered. The future region 26 may be shaded and/or colored differently to distinguish it from the historical data region 24 and may display a prediction by the user interface 10 as to the administration of drugs, and drug PK and PD graphs.

The user interface 10 may comprise user interface controls such as a time scale control 30, which may be a drop down menu, or may be a control button. A clinician could use the time scale control 30 to select a time duration displayed in time-based display of the graph region 23. The user interface 10 may also comprise time navigation buttons 32 for moving forward and backward with respect to time in the graph region 23. The clinician could manipulate the time navigation buttons 32 to view more of the historical data region 24 data for drugs administered previously, or to view more of the future region 26 data for a prediction further into the future of patient pharmacokinetic or pharmacodynamic state. Furthermore, the user interface 10 may comprise a refresh button 34 for refreshing the display of information on the user interface 10 to include newly updated information, alternatively the user interface 10 may simply refresh in real time, or at a fast enough refresh rate such that it is functionally equivalent to real time.

A clinician interacts with the user interface 10 during the provision of anesthesia to a patient and monitors the PK models for each of the drugs applied during the anesthesia and the PD models for the anesthetic effects of those drugs. The drug administration window 14 displays the documentation of any drugs that have been, or are being delivered to the patient in the anesthesia session. A clinician can document the administration of a drug by selecting a drug selector button 36 which may open a drug library (not depicted), which may allow the clinician to select from a list of available sedation, analgesia, and neuromuscular blocking (relaxant) drugs, in various standardized drug concentrations. The drugs are categorized within the drug library based upon the primary anesthetic effect of the drugs. However, it is understood that an anesthetic drug may have more anesthetic effects than just the primary anesthetic effect. The primary effect and any additional effects are worked into the PD models for these drugs. The drugs listed in the drug library may also be listed under both the generic name for the drug and the common name for the drug. Clinicians often use a drug's common name in practice and therefore are generally less familiar with the drug's generic name. As such, a drug library listing the drugs under both names improves the clinician's ability to properly find and select the drug that was administered.

Upon the selection of one of the drugs and a drug concentration from the drug library, the clinician may be prompted to enter how the drug is/was administered. This administration may be in the form of a bolus, such as from an injection, or delivered to the patient as an infusion, such as from a pump. The clinician may then have to enter the size of the bolus or the rate at which the drug infusion will be delivered. Alternatively, in the drug selection menu, the clinician may select that an infusion is to be supplied to the patient from an infusion pump that is capable of communicating with the user interface 10, such that the user interface 10 may receive a signal from the infusion pump representing the infusion concentration, infusion rate, and the start and end times of the infusion.

After this information has been entered, the drug name appears in the drug listing region 22 of the administration window 14 and a graphical time based indication of the drug administration appears on the graph region 23 of the drug administration window 14 at the time entered for the administration of the drug. For example referring to FIG. 1, 50 mg of the drug Propofol was administered at approximately 5:27 PM. As a result, the administration of the bolus is represented as a dot 40 at the time 5:27. The dot 40 is accompanied by the number 50 to represent the size (mg) of the bolus that was administered. Furthermore, an infusion of the drug Remifentanil was started at 5:28 PM. This is represented as a line 42 that extends from the start of the infusion until the infusion ends. The line 42 is accompanied by the number 55 to represent the infusion rate (ml/h) that is being administered.

Once the clinician has entered a first anesthetic drug, the clinician may repeat the process to document the administration of additional anesthetic drugs to the patient. Alternatively, the clinician may edit any of the drug administration documentations that have been made to correct any errors in documenting the delivery of a drug administration such as the drug type, drug concentration, or drug amount. Furthermore, the clinician can document and edit the time at which the drug was administered such that the clinician need not document the drug delivery information into the user interface 10 in real time, but rather can focus on delivering the drug to the patient, and then retroactively add in the documentation that that drug was administered.

When a clinician documents that a drug has been delivered at a particular time, the effect site concentration for the drug, or the PK graph, and the total anesthetic effect, or PD graph, are displayed in the graph region 23 of the window 12 that represents the primary effect of the drug, either sedation 16, analgesia 18, or neuromuscular block 20. As depicted in FIG. 1 Propofol has the primary effect of sedation therefore in the sedation window 14 the drug listing region 22 lists the drug name Propofol and the graph region 23 depicts a graph of Propofol PK graph 44. The graph region 23 of the sedation window 16 also displays the total sedation PD graph 46. The drug Remifentanil has a primary effect as an analgesic therefore, the drug listing region 22 of the analgesia window 18 displays the drug name Remifentanil and the graph region 23 depicts a Remifentanil PK graph 48. The graph region 23 of the analgesia window 18 further displays an analgesia PD graph 50.

The Propofol PK graph 44 displays the estimated effect site concentration of Propofol according to the Propofol PK model with respect to time. The effect site concentration of Propofol initially increases with the administration of the Propofol bolus 40. However, as the patient's body metabolizes the Propofol, the effect site concentration of Propofol decays over time.

The sedation PD graph 46 depicts the level of the pharmacodynamic effect that the patient is experiencing. Therefore as the effect site concentration of Propofol initially increases, the patient experiences a greater loss of consciousness. However, other anesthetic drugs that don't have a primary effect of sedation nonetheless have a sedative effect. Such is the case with Remifentanil. Therefore even after the patient's body begins to metabolize the Propofol, the Remifentanil infusion 42 initially furthers the patient's loss of consciousness. However, once the infusion of Remifentanil remains continuous the patient's loss of consciousness decreases as the Propofol is further metabolized.

The sedation PD graph 46 is normalized to a scale measuring total sedation against the percentage of the population that experiences a benchmark pharmacodynamic effect, in this case, the loss of consciousness. A first line 52 labeled “EC₅₀” indicates the level at which 50% of the population experiences a loss of consciousness due to the sedative effects of the drugs administered. The EC₉₅ marker 54 indicates the level at which 95% of the population experiences a loss of consciousness. The Propofol PK graph 44 is also normalized and displayed on this scale. The Propofol PK graph 44 is normalized to the effect site concentration required of Propofol alone to achieve a particular anesthetic effect.

The normalized scale, however, is not linear in its depiction; therefore, the graph region 23 further comprises a therapy window 56 that extends between the EC₅₀ line 52 and the EC₉₅ marker 54 to provide an indication of the non-linear scale. The non-linearity stems from the fact that it takes much more agent to get 95% of all patients to the desired effect, than it does to get 50% of patients. The therapy window 56 extends for the period at which the patient may experience a pharmacodynamic effect. The therapy window 56 may provide an indication of the non-linear scale for the PD effect between the EC₅₀ line 52 and the EC₉₅ line 54 in a variety of ways. As depicted, the therapy window 56 may comprise multiple distinct regions representing equal percentages of effectiveness range. In the example depicted in FIG. 1 the therapy window 56 is divided into three therapy window ranges, the 50%-65% region 58, the 65%-80% region 60, and the 80%-95% region 62. The clinician can note the non-linearity in the scale by simply viewing the area occupied by each of the therapy window regions. Alternatively, the therapy window may comprise more or fewer than three therapy window regions. Alternatively, the therapy window 56 may comprise a shaded gradient representing the percentile of the experienced effect.

Now referring to the analgesia window 18, the drug Remifentanil has the primary effect of being an analgesic therefore is listed in the drug listing region 22 of the analgesia window 18. An analgesia scale 38 indicates that the PK-PD scale 64 is selected for the analgesia window 18. Therefore, in the graph region 23 of the analgesia window 18 both the Remifentanil PK graph 48 and the total analgesia PD graph 50 are depicted. The scale on the left hand side of the graph region 23 indicates an EC₅₀ line 66 and an EC₉₅ marker 68 which denote the range for the therapy window 56 for the range in which 50%-95% of the population would not feel the pain associated with intubation. A second therapy window 70 is also displayed. The second therapy window 70 is indicative of a lower level of experienced analgesic effect. This analgesic effect is when the population would experience the proper level of post-op analgesia 72. A graph region 23 that displays multiple therapy windows, allows a clinician to further monitor the pharmacodynamic effect of the administered drugs.

However, in the graph region 23 of the analgesia window 18, the scale for the graph is normalized in the same manner as the scale for the sedation window 16. Therefore the Remifentanil PK graph 48 is difficult to read due to the relatively small dosage of Remifentanil administered to the patient with respect to the total analgesic pharmacodynamic effect depicted by analgesia PD graph 50. This is a common problem experienced with the display of an analgesic PK graph 48 and a analgesic PD graph 48 because the typical dosages of analgesic drugs are relatively small since the desired PD effect is produced in conjunction with a sedative.

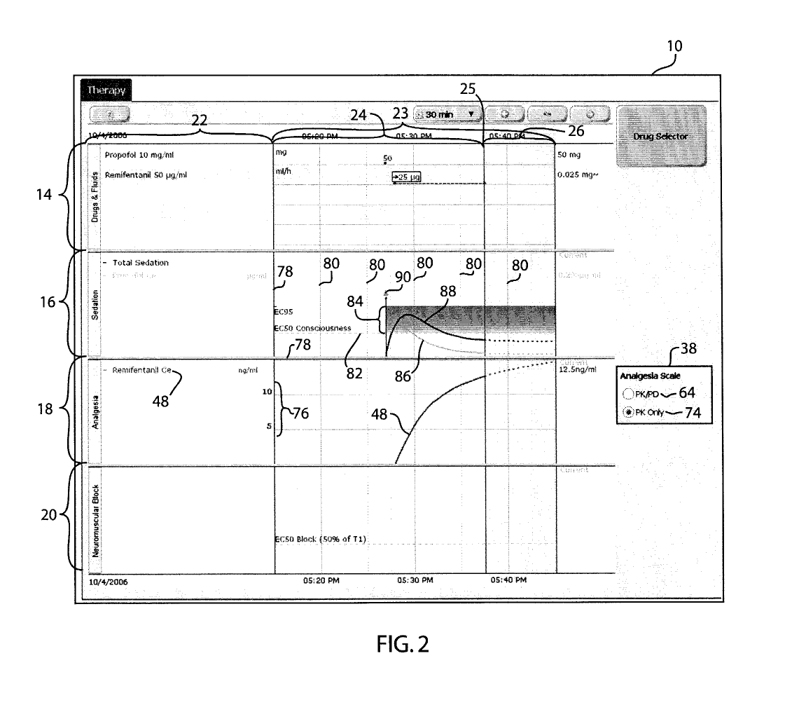

The clinician may improve the scale to better view the effect site concentration displayed by the Remifentanil PK graph 48 by selecting the PK only option 74 of the analgesia scale 38. As depicted in FIG. 2, the selection of the PK only option 74 removes the display of the analgesia PD graph 50 and changes the scaling of the analgesia window 18 to the display a numeric scale of Remifentanil effect site concentration 76.

Referring now to FIG. 2 the user interface 10 comprises all of the same drug administration data and sedation PK and PD graphs as depicted in FIG. 1, however; the analgesia scale 38 has been modified to the PK only option 74. In the drug listing region 22 of the analgesia window 18 the total analgesia indicator 50 has been removed and only the Remifentanil indicator 48 is depicted. The same is true in the graph region 23 wherein the total analgesia graph 50 has been removed and instead only a Remifentanil PK graph 48 is displayed. Since only the Remifentanil PK graph 48 is displayed, a better suited scale may be used to depict the effect site concentration. As depicted, a PK only scale 76 uses the concentration of nanograms per milliliter which results in a better scaled representation of the Remifentanil effect site concentration.

In an embodiment of the user interface, a clinician may switch back and forth between the PK/PD scale 64 and the PK only scale 74 in order to receive information regarding the patient's total analgesia, or a more detailed view of the effect site concentration of any analgesic drugs that have been administered to the patient.

In an embodiment of the user interface it is important that the graphs are drawn in such a way that the most key information is the most visible on the graph. Therefore, a specific hierarchy of the graph elements may be followed such that any previously drawn element is replaced by any future element if the drawing of both elements conflicts. One such hierarchy that may be used in an embodiment, as described in relation to the graph region 23 of the sedation window 16 in FIG. 2, may be as follows. First the graph X and Y axes 78 would be drawn. Next the graph grid lines 80, denoting time intervals, would be drawn. Next the shading for the background of the future region 26 would be drawn. Next, the EC₅₀ line 82 would be drawn. Then for each point in time, from past through the future, the therapy windows 84 would be drawn. Next, any drug PK graphs 86 followed by the PD graphs 88. Then, any specific messages and/or symbols are displayed, such as an interactions valid symbol 90 that indicates that all of the drugs that have been documented are drugs for which the user interface has a valid PK and PD model. Finally, any time independent messages, such as error messages (not depicted) are displayed.

An embodiment of the user interface may comprise that the graphs in the graph region 23, including any drug infusions, pharmacokinetic graphs, and pharmacodynamic graphs are displayed and updated in real time as the clinician monitors the display for information regarding the patient's depth of anesthesia.

In a further embodiment, the same PK and PD graphs as depicted and described in reference to the analgesia window 18 and the sedation window 16 are displayed in relation to the neuromuscular block window 20 when the clinician documents the administration of a neurological blocking drug to the patient.

In a still further embodiment, despite a drug being labeled as having a primary effect towards sedation, analgesia, or neuromuscular blocking, the drug may have additional effects in one of the other anesthetic effect categories. This may result in producing either an additive effect in the total sedation, total analgesia, or total neuromuscular blocking, or the combination of drugs that have been applied to the patient may produce a combinational effect greater than a predicted additive effect.

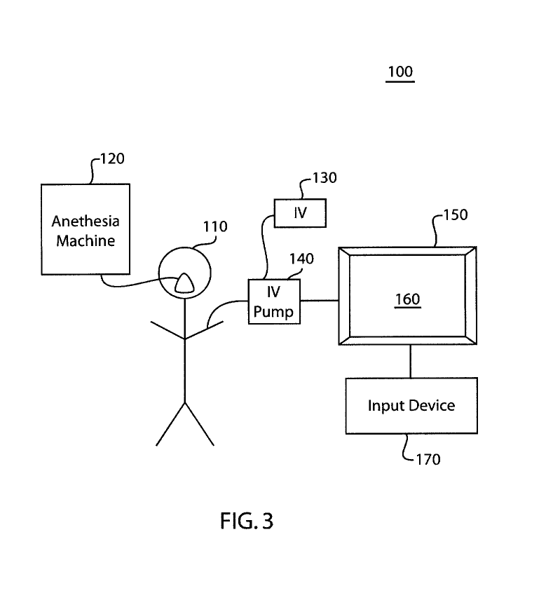

FIG. 3 depicts a schematic diagram of an embodiment of a system, such as a critical care system 100. The critical care system 100 comprises a patient 110. The patient 110 may be receiving an anesthetic agent from an anesthesia delivery machine 120, or via an intravenous (IV) drug delivery system 130. Additionally, the IV drug delivery system 130 may be connected to an IV pump 140. The IV pump 140 may be used to control the rate and amount of the drug delivered to the patient 110 by the IV drug delivery system 130. If the IV pump 140 is able to communicate with a display 150, the display 150 can receive IV drug administration data from the IV pump 140. The display 150 comprises a user interface 160 that displays drug administration data and PK and PD models to a clinician. Drug administration data not received by the display 150 from the IV pump 140 must be entered manually by the clinician. The clinician may enter the drug administration data into the display 150 using an input device 170. The input device 170 may be an external input device 170, such as a keyboard. Alternatively, the input device may be an input device that is integral with the display 150, such as a touch screen.

Embodiments of the user interface provide advantages over the prior art in that the clinician is presented with a single user interface that displays both the pharmacokinetic and pharmacodynamic information on a single graph. Furthermore, embodiments of the user interface provide the advantage of allowing a clinician greater flexibility in the time at which the clinician must enter any drug administration data. By allowing the clinician to enter drug administration data retroactively, the need for an extra clinician in an operating room to simply document the delivery of drugs to the patient in real time may be eliminated, thus reducing the number of clinicians that must be present in the already typically crowded operating room. Furthermore, embodiments of the user interface make it easier for the clinician to interpret the pharmacodynamic graphs displayed on the user interface due to the therapy window including indications for ranges of the effect experienced by patients such that the clinician is aware of the non-linearity of the pharmacodynamic effect scale. Improved ease in interpreting PK and PD graphs by a clinician can result in the clinician having an improved sense of the patient's depth of anesthesia, resulting in improved patient care.

This written description uses examples to disclose the invention, including the best mode, and also to enable any person skilled in the art to make and use the invention. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

Various alternatives are contemplated as being with in the scope of the following claims, particularly pointing out and distinctly claiming the subject matter regarded as the invention.

We claim:

1. A graphical user interface, the graphical user interface comprising:
a drug library comprising data related to a plurality of anesthetic drugs;
a drug window for displaying drug administration data of a drug selected from the drug library;
at least one window separate from the drug window, the at least one window being disposed for the display of a pharmacokinetic graph and a pharmacodynamic graph of a drug selected from the drug library; and
a therapy window associated with at least one window separate from the drug window the therapy window comprising an indication of the non-linearity of the pharmacodynamic.

2. The graphical user interface of claim 1, wherein the data related to a plurality of anesthetic drugs comprises a generic name and a common name for each drug.

3. The graphical user interface of claim 2, wherein the drug window displays the generic name for the drug.

4. The graphical user interface of claim 3, wherein the drug window displays the amount of the drug administered to the patient and the type of drug administration.

5. The graphical user interface of claim 1, further comprising a sedation model window, an analgesia model window, and a neuromuscular block model window.

6. The graphical user interface of claim 5, further comprising a scale selector comprising a first condition wherein the pharmacokinetic graph and the pharmacodynamic graph are displayed and a second condition where only the pharmacokinetic graph is displayed.

7. The graphical user interface of claim 6, wherein the scale selector means only controls the display of the analgesia model window.

8. The graphical user interface of claim 1 wherein the indication of non-linearity comprises more than one probability range.

9. The graphical user interface of claim 1 wherein the indication of non-linearity comprises a shaded gradation based upon probability.

10. A method of displaying anesthetic drug models to a clinician, the method comprising the steps of:
receiving a selection of a drug from a drug library to be documented;
receiving drug administration data for the selected drug;
displaying the drug administration data in a drug window;
displaying a pharmacokinetic model and a pharmacodynamic model for the selected drug;
displaying a therapy window capable of identifying a non-linear scale for the pharmacodynamic model.

11. The method of claim 10, wherein the drug library comprises drug principal effect data for each drug in the drug library.

12. The method of claim 11, wherein the principal effect data is selected from the list of sedation, analgesia, and neuromuscular block effects.

13. The method of claim 12, further comprising the steps of:
receiving an indication that a scale selector is in a first condition;
displaying the pharmacokinetic model and the pharmacodynamic model in the analgesia window; and
displaying the therapy window in the analgesia window.

14. The method of claim 13, further comprising the steps of:
receiving an indication that the scale selector is in a second condition;
ending the display of the pharmacodynamic model and the therapy window.

15. The method of claim 10 further wherein the step of displaying the pharmacokinetic model and the pharmacodynamic model for the selected drug further comprises the steps of:
drawing at least one graph axis line;
drawing at least one graph grid line;
shading the background of a future region;
drawing an EC50 line;
drawing the therapy window;
drawing the pharmacokinetic model;
drawing the pharmacodynamic model; and
displaying any messages.

16. The method of claim 15 wherein the steps for the step of displaying the pharmacokinetic model and the pharmacodynamic model are performed in the order that the steps are listed.

17. A graphical user interface for use by a clinician in the monitoring of the depth of anesthesia of a patient, the graphical user interface comprising:
a drug window for displaying a plurality of drug administration data;
a sedation model window disposed for the display of a pharmacokinetic graph and a pharmacodynamic graph associated with the drug administration data, the sedation model window further comprising a sedation therapy window associated with the pharmacodynamic graph, the sedation therapy window comprising an indication of the non-linearity of the pharmacodynamic graph;
an analgesia model window disposed for the display of a pharmacokinetic graph and a pharmacodynamic graph associated with the drug administration data, the analgesia model window further comprising an analgesia therapy window associated with the pharmacodynamic graph, the analgesia therapy window comprising an indication of the non-linearity of the pharmacodynamic graph; and
a scale selector associated with the analgesia model window, the scale selector comprising a first condition wherein the pharmacokinetic graph and the pharmacodynamic graph are displayed and a second condition where only the pharmacokinetic graph is displayed.

18. The graphical user interface of claim 17, further comprising a neuromuscular block window disposed for the display of a pharmacokinetic graph and a pharmacodynamic graph associated with the drug administration data, the neuromuscular model window further comprising a neuromuscular therapy window associated with the pharmacodynamic graph, the neuromuscular therapy window comprising an indication of the non-linearity of the pharmacodynamic graph.

19. The graphical user interface of claim 17, wherein the indication of the non-linearity of the pharmacodynamic graph comprises a plurality of probability ranges.

20. The graphical user interface of claim 17, wherein the drug window displays the amount of the drug administered to the patient and the type of drug administration.
